(19) United States
(12) Patent Application Publication
(10) Pub. No.: US 2023/0326564 A1
(43) Pub. Date: Oct. 12, 2023

(54) MEDICAL INFORMATION PROCESSING APPARATUS, MEDICAL INFORMATION PROCESSING METHOD AND NON-TRANSITORY COMPUTER-READABLE MEDIUM

(71) Applicant: Canon Medical Systems Corporation, Otawara-shi (JP)

(72) Inventors: Masahiro YOSHIWAKI, Nasushiobara (JP); Takahiko NISHIOKA, Otawara (JP)

(73) Assignee: Canon Medical Systems Corporation, Otawara-shi (JP)

(21) Appl. No.: 18/192,798

(22) Filed: Mar. 30, 2023

(30) Foreign Application Priority Data
Mar. 31, 2022 (JP) 2022-059273

(51) Int. Cl. G16H 10/60; G16H 50/20

(52) U.S. Cl. CPC G16H 10/60; G16H 50/20; G16H 50/70

(57) ABSTRACT

According to one embodiment, a medical information processing apparatus includes processing circuitry. The processing circuitry acquires patient information relating to a target patient, environment information relating to an environment for execution, and target data relating to the target patient. processing circuitry determines, from a plurality of trained models differing from each other in processing speed and accuracy, a combination of trained models that satisfies a condition according to the patient information and the environment information.

### CROSS-REFERENCE TO RELATED APPLICATIONS

This application is based upon and claims the benefit of priority from Japanese Patent Application No. 2022-059273, filed Mar. 31, 2022, the entire contents of which are incorporated herein by reference.

### FIELD

Embodiments described herein relate generally to a medical information processing apparatus, a medical information processing method and a non-transitory computer-readable medium.

### BACKGROUND

With the recent development of medical image diagnosis through a deep learning technology, the accuracy of diagnostic processing using a machine-trained model has increased. A machine-trained model, however, takes a tremendous amount of processing time when processing a higher-definition image. On the other hand, reducing the processing time will degrade the accuracy. Especially in medical settings, there is a case where urgency is required such as an emergency and a case where highly accurate results are required.

### BRIEF DESCRIPTION OF THE DRAWINGS

FIG. 1 is a block diagram showing a medical information processing system according to an embodiment.

FIG. 2 is a block diagram showing details of a medical information processing apparatus according to the embodiment.

FIG. 3 is a flowchart showing an operation of the medical information processing apparatus according to the embodiment.

FIG. 4 is a diagram showing an overview of the variations of a trained model.

FIG. 5 is a diagram showing an example of a user interface indicating priority to speed or priority to accuracy.

FIG. 6 is a diagram showing a first example of a user interface observed after analytical processing is performed.

FIG. 7 is a diagram showing a second example of a user interface observed after analytical processing is performed.

FIG. 8 is a diagram showing a third example of a user interface observed after analytical processing is performed.

FIG. 9 is a conceptual diagram showing a training process of a set model.

FIG. 10 is a diagram showing an example of a suggestion made by a set model.

### DETAILED DESCRIPTION

In general, according to one embodiment, a medical information processing apparatus includes processing circuitry. The processing circuitry acquires patient information relating to a target patient, environment information relating to an environment for execution, and target data relating to the target patient. processing circuitry determines, from a plurality of trained models differing from each other in processing speed and accuracy, a combination of trained models that satisfies a condition according to the patient information and the environment information.

Hereinafter, a medical information processing apparatus, a medical information processing method, and a storage medium storing a medical information processing program according to the embodiment will be described with reference to the accompanying drawings. In the embodiment described below, elements assigned the same reference numeral perform the same operation, and repeat descriptions will be omitted as appropriate. Hereinafter, the embodiment will be described with reference to the accompanying drawings.

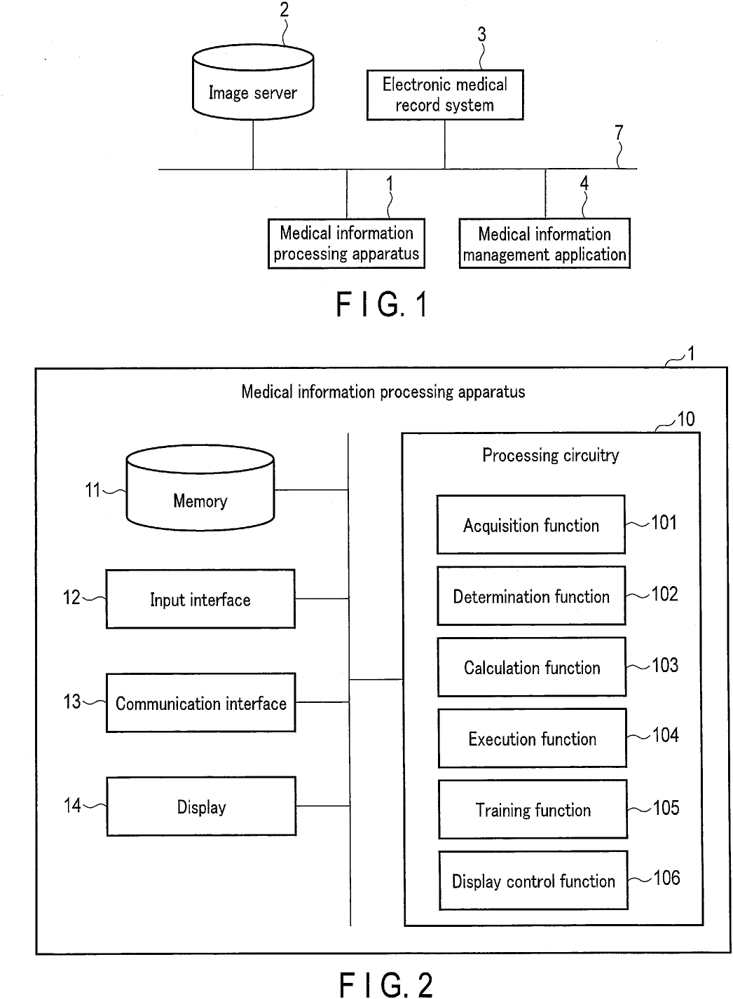

A medical information processing system according to the embodiment will be described with reference to FIG. 1.

The medical information processing system according to the embodiment includes a medical information processing apparatus 1, an image server 2, an electronic medical record system 3, and a medical information management application 4. The medical information processing apparatus 1, the image server 2, the electronic medical record system 3, and the medical information management application 4 are connected to one another via a network 7. Although the medical information processing apparatus 1 according to the embodiment is assumed to be separate from the image server 2, the electronic medical record system 3, and the medical information management application 4, the medical information processing apparatus 1 may be included in at least any one of the image server 2, the electronic medical record system 3, or the medical information management application 4.

The medical information processing apparatus 1 selects, among the plurality of trained models, a combination of trained models having speed and accuracy that satisfy the needs of a user, and performs data processing on target data. The medical information processing apparatus 1 also displays the selected combination of trained models and information relating to the speed and the accuracy of each trained model. The medical information processing apparatus 1 will be detailed later with reference to FIG. 2.

The image server 2 is, for example, a picture archiving and communication system (PACS), and is a system that stores medical image data and manages the stored medical image data. The image server 2 stores and manages medical image data converted in accordance with, for example, the digital imaging and communications in medicine (DICOM) standard.

The electronic medical record system 3 is a system that stores electronic medical record data including patient information, etc., and manages the stored electronic medical record data. The patient information includes information relating to, for example, a patient ID and a patient’s name, gender, age, past medical history, lifestyle habit, etc., and further includes information concerning an electronic medical record, such as finding information, disease name information, vital sign information, examination stage information, a clinical pathway, and information on details of treatment. The clinical pathway refers to a time-series standard treatment plan.

The medical information management application 4 is an application that is capable of integrating and managing patient’s clinical records such as treatment information and examination information in chronological order, so that the clinical records can be shared by doctors or by users represented by medical staff such as a doctor, a technician, and a nurse.

The network 7 is, for example, an intra-hospital network. The connection realized by the network 7 may be either wired or wireless. The connection need not necessarily be made to an intra-hospital network, provided that the security is ensured. For example, the connection may be made to a public communication line such as the Internet via a virtual private network (VPN), etc.

Next, the medical information processing apparatus 1 will be detailed with reference to the block diagram shown in FIG. 2.

The medical information processing apparatus 1 shown in FIG. 2 includes processing circuitry 10, a memory 11, an input interface 12, a communication interface 13, and a display 14. The processing circuitry 10, the memory 11, the input interface 12, the communication interface 13, and the display 14 are connected to one another via, for example, a bus so as to be able to communicate with one another.

The processing circuitry 10 is a processor that functions as the center of the medical information processing apparatus 1. The processing circuitry 10 includes an acquisition function 101, a determination function 102, a calculation function 103, an execution function 104, a training function 105, and a display control function 106.

The acquisition function 101 acquires patient information relating to a target patient, environment information relating to an environment for execution, and target data relating to the target patient. The environment information includes at least one of information relating to the system environment for executing analytical processing using a trained model or information relating to the scale of a hospital. The system environment includes, for example, information relating to environment for execution such as the number of workstations that execute analytical processing using a trained model, the central processing unit (CPU) of the workstations, and the specifications of the memory. The scale of a hospital includes information such as the type of facility of the hospital, the number of medical staff members such as doctors, and the types of clinical departments.

The determination function 102 determines, from a plurality of trained models differing from each other in speed and accuracy, a combination of trained models that satisfies conditions according to the patient information and the environment information.

The calculation function 103 calculates a processing time and estimation accuracy that are assumed for the trained models.

The execution function 104 performs analytical processing using the determined combination of trained models. Also, the execution function 104 inputs patient information, environment information, and target data, all of which are objects to be processed, to a trained set model (which will be described later), and thereby outputs a combination of trained models.

The training function 105 trains a network model using information regarding the trained models included in the combination, the patient information, the environment information, and the target data as input data, and using feedback information relating to feedback from a user on at least of one of a state during execution of the analytical processing using the combination, or an analysis result, and generates a trained set model.

The display control function 106 causes the display 14 or the like to display the information on the trained models.

The memory 11 is a storage device storing various kinds of information, such as a ROM (Read Only Memory), a RAM (Random Access Memory), an HDD (Hard Disk Drive), an SSD (Solid State Drive), an integrated-circuit storage device, or the like. The memory 11 may be, for example, a CD-ROM drive, a DVD drive, or a drive which reads and writes various kinds of information from and to a portable storage medium such as a flash memory. The memory 11 need not necessarily be realized by a single storage device. For example, the memory 11 may be realized by multiple storage devices. The memory 11 may be provided in another computer that is connected to the medical information processing apparatus 1 via the network 7.

The memory 11 stores various items including a processing program according to the embodiment. For example, this program may be pre-stored in the memory 11. In another exemplary implementation, the program may be distributed as an item stored in a non-transitory storage medium, and then read from the non-transitory storage medium to be installed in the memory 11.

The input interface 12 receives various kinds of input operations from a user, converts the received input operations into an electric signal, and outputs the electric signal to the processing circuitry 10. The input interface 12 according to the embodiment is connected to one or more input devices such as a mouse, a keyboard, a trackball, a switch, a button, a joystick, a touchpad, and a touch panel which allows input of instructions through touch on its operation screen. The input devices coupled to the input interface 12 may each be an input device arranged at an external computer connected via a network, etc.

The communication interface 13 performs data communication with the image server 2, the electronic medical record system 3, and the medical information management application 4, as well as a hospital information system and a radiation section information system (not shown). For example, the communication interface 13 performs data communication according to a preset known protocol. For example, communications complying with health level 7 (HL7) are performed between the medical information processing apparatus 1 and each of the hospital information system, the electronic medical record system 3, the medical information management application 4, and the radiation section information system. Also, communications complying with, for example, digital imaging and communications in medicine (DICOM) are performed between the medical information processing apparatus 1 and each of the image server 2 and the medical information management application 4.

The display 14 displays graphical user interfaces (GUIs), etc., for accepting various operations from users. The display may be any display such as a cathode ray tube (CRT) display, a liquid crystal display, an organic EL display, an LED display, a plasma display, or a touch display which allows for touch input operations. The medical information processing apparatus 1 may not be furnished with the display, and may instead cause an external display to display GUIs or cause GUIs to be displayed via a projector or the like.

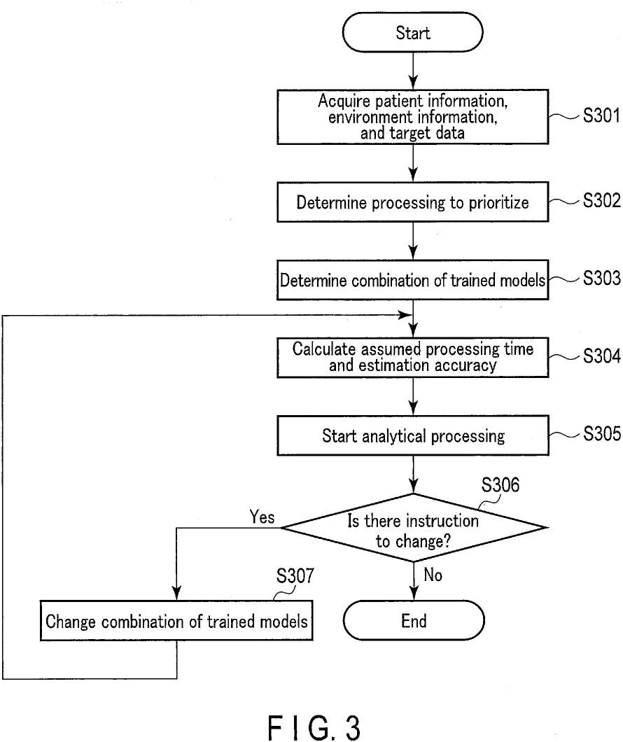

Next, an example of an operation of the medical information processing apparatus 1 according to the embodiment will be described with reference to the flowchart shown in FIG. 3. This operation example assumes sequential performance of a plurality of analytical processing steps and obtainment of a processing result for a single task. This operation example also assumes a case where a plurality of trained models are prepared in advance for each of the analytical processing steps.

In step S301, the acquisition function 101 acquires patient information, environment information, and target data to be processed. Herein, the target data is assumed to be a medical image, but may be time-series data such as vital data of heart rate, blood pressure, and the like.

In step S302, the determination function 102 determines whether to prioritize the speed or the accuracy when performing analytical processing by applying machine learning to the target data. Whether to prioritize the speed or the accuracy may be determined based on a user’s instructions, for example, or determined according to whether or not various kinds of information and data satisfy the determination conditions based on the patient information, environment information, and target data acquired in step S301. For example, in the case of an urgent patient taken by ambulance to hospital, it is often the case that the processing speed should be prioritized; thus, if it can be determined from the patient information that the case is an emergency, it may be determined that the speed should be prioritized. On the other hand, if one desires to refer to a result of the output from the trained model as reference information for the findings, for example, it is often the case that the estimation accuracy should be prioritized; thus, if the target data is given supplementary information indicating that it is for reference information, for example, it may be determined that the estimation accuracy should be prioritized when this supplementary information is present.

In step S303, the determination function 102 selects, based on the priority determined in step S302, one trained model from among a plurality of trained models prepared for each analytical processing step, and determines a combination of trained models for a series of analytical processing steps. For example, if the speed is to be prioritized, the determination function 102 may select, for each of the analytical processing steps, a trained model that requires the shortest processing time. Likewise, if the accuracy is to be prioritized, the determination function 102 may select, for each of the analytical processing steps, a trained model that has the highest estimation accuracy.

In step S304, the calculation function 103 calculates a processing time and estimation accuracy of the trained models assumed in each of the analytical processing steps.

In step S305, the analytical processing on the target data is started, and the display control function 106 causes, for example, the display 14 to display a user interface relating to the state of the analytical processing.

In step S306, the execution function 104 determines whether or not there is an instruction from a user to change, herein, the priority from the speed to the accuracy or from the accuracy to the speed. If an instruction from a user to change the priority is received, the process proceeds to step S307, and if there is no instruction from a user to change the priority, the analytical processing is continued until there is an instruction from a user to change the priority.

In step S307, the combination of trained models is changed based on the instruction to change the priority. For example, the combinations of trained models determined for the currently performed analytical processing and the subsequent analytical processing are changed. Alternatively, the trained models for the currently performed analytical processing are not changed, and the combinations of trained models determined for the next analytical processing and the subsequent analytical processing are changed. After these combinations are changed, the process returns to step S304, and the same process is repeated.

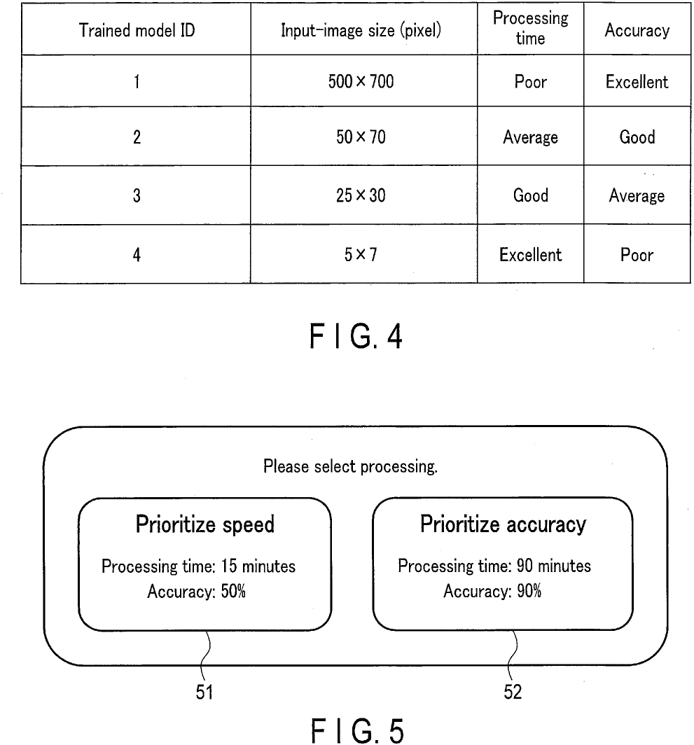

Next, an overview of the variations of the trained models assumed in the embodiment will be described with reference to FIG. 4.

FIG. 4 is a table showing model information of a plurality of trained models that perform a single analytical processing step. In this instance, four different trained models are shown, and pieces of information relating to an input-image size, processing time, and accuracy are stored in such a manner that they are associated with each other. The input-image size indicates the resolution (the number of horizontal × vertical pixels) of an image that can be processed. The processing time indicates the ranking of the trained models relating to the processing time. The accuracy indicates the ranking of the trained models relating to the estimation accuracy. Herein, it is indicated that the performance degrades in the order of “Excellent”, “Good”, “Average”, and “Poor”.

Specifically, the trained model ID “1”, the input-image size “500 × 700” pixels, the processing time “Poor”, and the accuracy “Excellent”, for example, are associated with each other. That is, it is indicated that the model of the trained model ID “1” is a model that takes a long processing time but has a high estimation accuracy. On the other hand, it is indicated that the trained model ID “4”, which has an input-image size of “5 × 7” pixels, a processing time of “Excellent”, and an accuracy of “Poor”, has a low estimation accuracy but exhibits a fast processing time.

An example of a user interface which allows a user to select whether the speed should be prioritized or the accuracy should be prioritized in step S302 will be described with reference to FIG. 5.

FIG. 5 is a window for allowing a user to select whether processing prioritizing the speed or processing prioritizing the accuracy should be performed, as a user interface caused to be displayed by the display control function 106. If the processing speed is to be prioritized, a speed-prioritized button 51 is pressed. If the estimation accuracy is to be prioritized, an accuracy-prioritized button 52 is pressed.

The action of “pressing a button” as described in the embodiment includes: pressing a physical button with a setting assigned to it; selecting a button, such as the speed-prioritized button 51, with a mouse cursor or the like, and clicking the button; and touching a button area if the display is a touch panel display.

The manner of display is not limited to display through a window as shown in FIG. 5, and any display manner such as allowing a user to select a character string may be adopted. Also, the medical information processing apparatus 1 according to the embodiment may receive selection of a priority not only through input to an input device or a touch panel display but also through voice input using a microphone. That is, as long as designation of a priority by a user can be received, any acquisition manner may be adopted.

Also, in an emergency case and the like, the speed may be mandatorily prioritized, and in this case the accuracy-prioritized button 52 may be hidden or set so as to not be selected.

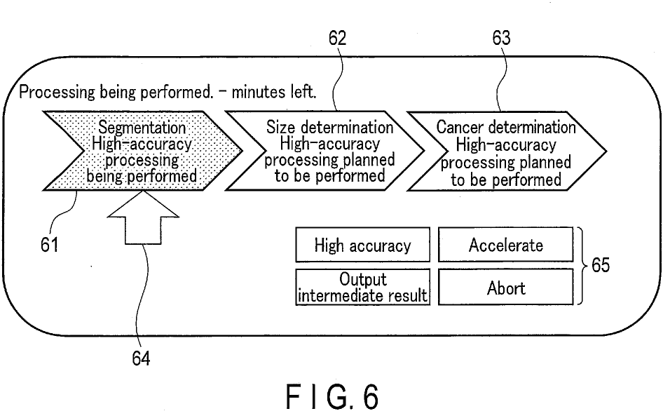

Next, a first example of a user interface caused to be displayed by the display control function 106 after the analytical processing in step S305 is started will be described with reference to FIG. 6.

FIG. 6 assumes the case where three analytical processing steps are performed and a processing result for a single task is thereby obtained. This case assumes a task of determining the benignancy or malignancy of a tumor, and shows an example of performing the following analytical processing steps in the mentioned order: a first analytical processing step 61 of performing segmentation of the tumor; a second analytical processing step 62 of identifying the size of the tumor; and a third analytical processing step 63 of determining whether the tumor is benign or malignant, in other words, whether the tumor is cancer or not.

The stage of the progression of the processing may be represented by a cursor 64, wherein the state in which the processing has been completed up to the middle of the first analytical processing step 61 of performing segmentation is shown by the cursor 64. This makes it easy to know, with regard to the task, which analytical processing step is currently being performed and how much the processing has progressed.

Also, a switch may be made as to whether to set to “prioritize the accuracy” or set to “prioritize the speed” for each of the analytical processing steps by using a button group 65. For example, if a “High accuracy” button is pressed, a trained model designed for prioritizing the accuracy is applied to the currently proceeding analytical processing step and the subsequent analytical processing steps or to the analytical processing steps that follow the currently proceeding analytical processing step. On the other hand, if an “Accelerate” button is pressed, a trained model designed for prioritizing the speed is applied to the currently proceeding analytical processing step and the subsequent analytical processing steps or to the analytical processing steps that follow the currently proceeding analytical processing step. As a matter of course, the wording presented on the buttons is not limited to the example shown in FIG. 6; any wording may be used, provided that a user can select a setting to prioritize the accuracy and a setting to prioritize the speed.

While the analytical processing is proceeding based on the setting to prioritize the speed, it may be impossible, depending on the resolution of the image being processed, to change the setting to prioritize the accuracy in the middle of the processing once the resolution is lowered. In such a case, a setting such as disallowing pressing of the “High accuracy” button or hiding the “High accuracy” button may be made for the processing that cannot prioritize the accuracy.

Also, the calculation function 103 may recalculate the remaining estimated processing time to display it.

Also, intermediate data obtained in the middle of the analytical processing may be displayed by pressing an “Output intermediate result” button. For example, if convolutional processing for extracting features is performed on a medical image as the analytical processing, intermediate data, a heat map relating to feature extraction, or the like obtained after the performance of the convolutional processing may be displayed. By pressing an “Abort” button, the processing using the trained models may be aborted at the timing when the button is pressed or after the analytical processing that is being performed is terminated, so that the processing result is output. For example, if a doctor wishes to perform processing up to the step of determining the size of a tumor by using the trained models and then perform the subsequent step of “determining whether it is cancer” by him/herself, s/he can abort the processing through the “Abort” button, and perform the cancer determination in the state where the location and the size of the tumor have been analyzed.

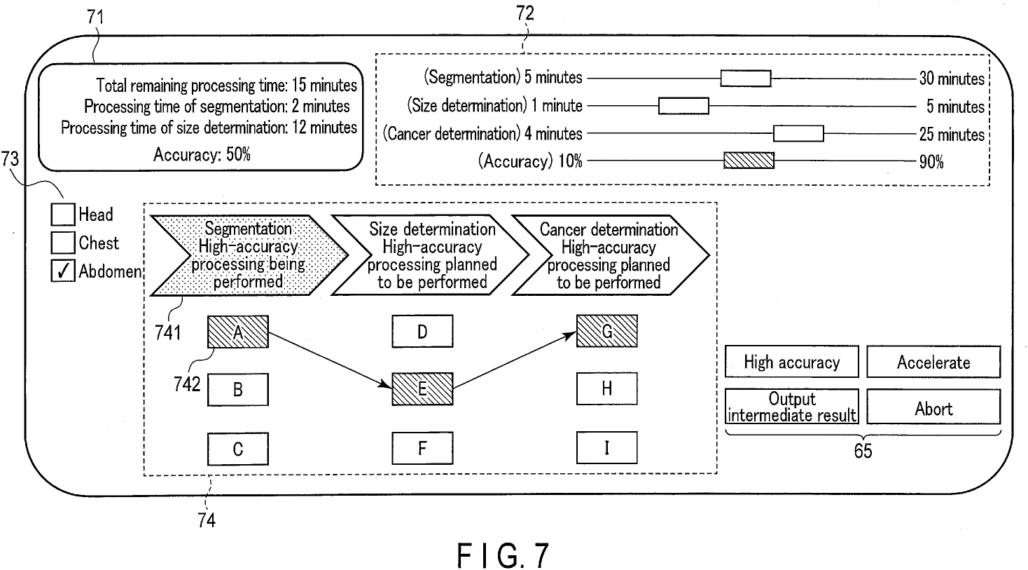

Next, a second example of a user interface caused to be displayed by the display control function 106 will be described with reference to FIG. 7.

FIG. 7 shows an example in which more detailed information such as a remaining time of each analytical processing step is displayed in addition to the user interface shown in FIG. 6.

A processing-time window 71 displays a total remaining processing time and a remaining processing time of each analytical processing step. Information on the accuracy of the entire task, which includes the respective analytical processing steps, may also be displayed. The accuracy may be presented by using an average value of the accuracy of the respective analytical processing steps or other statistical means such as a weighted average of the accuracy obtained by weighting the respective analytical processing steps.

A slide-bar window 72 displays slide bars relating to the processing time of each analytical processing step and the overall accuracy of the task. To give a specific example, if a user moves the slide bar of “Segmentation” in a direction toward a shorter time, a trained model designed to prioritize the speed is applied, whereas if a user moves the slide bar in a direction toward a longer time, a trained model designed to prioritize the accuracy is applied. If the processing time is changed by adjusting the slide bar relating to the processing time of each analytical processing step, the overall accuracy of the task is recalculated by the calculation function 103, and the slide bar of “accuracy” is displayed in tandem therewith in a position corresponding to the value obtained by the recalculation.

Also, the slide bar of “accuracy” may be adjusted to thereby move the slide bar of each analytical processing step in tandem therewith. For example, if a user moves the slide bar in a direction toward higher accuracy, the slide bar of each analytical processing step moves in tandem therewith in a direction toward a longer processing time. In this manner, displaying the slide-bar window 72 in addition to or in place of the button group 65 allows for more detailed setting of the processing time and the estimation accuracy.

The adjusted values of the slide bars described above are set in stages according to the number of trained models. For example, in the case of the analytical processing step of “Segmentation”, since three trained models are prepared, the slide bar of the processing time can be adjusted in three stages.

A target-site window 73 is a check box indicating a target site to be processed. For example, if there is a target site that is desired to be processed among a plurality of target sites included in a captured medical image, a check box of the target site concerned may be checked.

A progress-status window 74 includes an icon 741 indicating which processing among the analytical processing steps is being performed and an icon 742 indicating a plurality of trained models capable of performing the respective analytical processing steps. In the example shown in FIG. 7, the progress-status window 74 highlights the trained models that are used in the respective analytical processing steps and connects them with an arrow according to the progress of the analytical processing. Specifically, a trained model “A” with regard to the analytical processing step of “Segmentation”, a trained model “E” with regard to the analytical processing step of “size determination”, and a trained model “G” with regard to the analytical processing step of “cancer determination” are connected to each other. Such a display makes it possible to understand at a glance which trained model is being used. Also, each of the icons 741 may be displayed in different display manners such as by using different colors based on whether the speed is prioritized or the accuracy is prioritized.

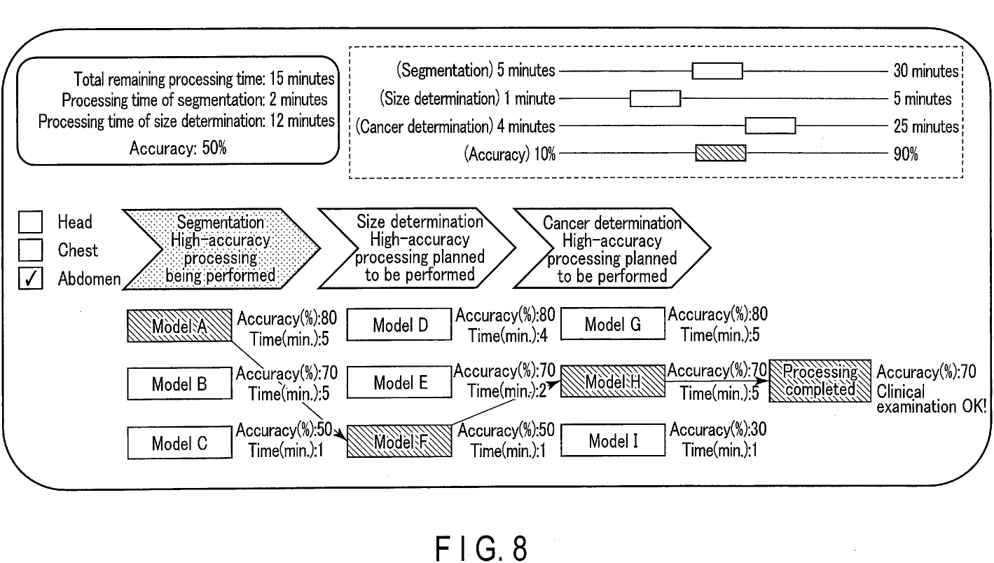

Next, a third example of a user interface caused to be displayed by the display control function 106 will be described with reference to FIG. 8.

FIG. 8 shows an example in which the estimation accuracy (indicated as “accuracy” in FIG. 8) and the processing time (indicated as “time” in FIG. 8) are displayed for each of the trained models that can be used in the respective analytical processing steps. In this manner, a user can easily know which trained model is being selected in each analytical processing step and can also know the estimation accuracy and the processing time of each trained model. A user may also be allowed to select a trained model different from the currently set trained model and thereby utilize the newly selected trained model for the analytical processing. In addition, if there is a model that cannot be utilized in the analytical processing step subsequent to the currently proceeding analytical processing step in light of combination with the model utilized in the currently proceeding analytical processing step, such a model may be displayed with a broken line or in a light color so that it cannot be selected.

Based on the feedback information relating to a user’s action such as switching to a trained model designed to prioritize the speed while the analysis prioritizing the accuracy, in which the setting to prioritize the accuracy is selected, is being performed, a combination of trained models may be trained to generate trained models for presenting a combination of trained models for processing the task.

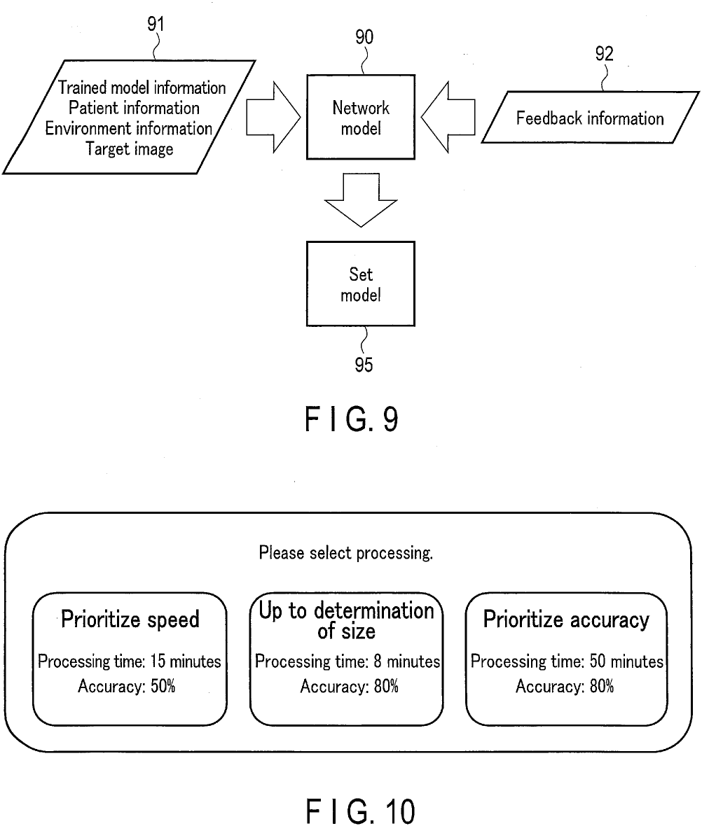

A training process of a set model for selecting a trained model suitable for a task will be described with reference to the conceptual diagram shown in FIG. 9.

FIG. 9 shows an example of input data and output data for training a network model 90.

The training function 105 trains a network model 90 using the information on the trained models used in the analytical processing, patient information, environment information, and target image as input data 91, and using feedback information on the state of the input data 91 as ground truth data 92.

The trained model information includes information regarding the identifier, estimation accuracy, processing time of the trained model used in the analytical processing, and information regarding the estimation accuracy and processing time of the whole task. The feedback information indicates a user’s action relating to the state of the input data. Examples of the feedback information include: whether or not the estimation processing of the task performed by the trained model has been terminated in the middle of the processing; whether or not the priority has been switched (i.e., whether the priority has been changed from the accuracy to the speed or the priority has been changed from the speed to the accuracy); a combination of trained models selected; and an evaluation of the processing result of the task (e.g., ratings such as excellent, good, average, fail, etc.).

A set model 95, which is a trained model that has learned the feedback given by a user on the execution of the trained model used in the analytical processing, is generated by training the network model 90 using the above-described input data 91 and ground truth data 92.

According to the set model 95, a task with accuracy and processing time according to the user’s preference, that is, a combination of trained models, can be suggested.

Next, an example of a suggestion made by the set model 95 will be described with reference to FIG. 10.

FIG. 10 shows a user interface similar to that shown in FIG. 5, and the user interface shown in FIG. 10 is a user interface obtained when the set model 95 is used and relates to a selection screen for selecting whether to prioritize the speed or prioritize the accuracy. Note that FIG. 5 assumes an example of the case where the set model 95 is not used. Also, the feedback information used as the ground truth data 92 assumes examples including many cases where after performing setting to prioritize the accuracy, a user selects a trained model for the analytical processing so as to prioritize the speed in the middle of the processing and also aborts the execution of the task in the middle of the processing (herein, in the stage where the second analytical processing step of determining the size of the tumor is completed).

The execution function 104 inputs the patient information, environment information, and target data, all of which are objects to be processed, to the set model 95, and thereby outputs a combination of trained models for processing the task.

As shown in FIG. 10, since training to prioritize the speed has been performed, a combination of trained models for each analytical processing step will be selected via the set model 95 so that the processing time will be short while the accuracy is prioritized. Also, for the analytical processing performed with the speed prioritized, a combination of trained models changed so as to reduce the processing time may be presented. In addition, a new button indicating “Up to determination of size of the tumor” may be generated and displayed, as shown in FIG. 10.

As described above, according to the set model, a combination for the analytical processing according to the user’s preference can be presented.

According to the embodiment described above, one trained model is selected from among a plurality of trained models for each analytical processing step according to whether the speed should be prioritized or the accuracy should be prioritized based on the patient information, environment information, and target data, whereby a combination of trained models is determined. Thus, optimal processing responding to needs can be provided. Also, with the feedback result based on at least one of the result of the user’s selection of the priority or the result of the selection of the trained models, an optimal combination of trained models can be presented by using a trained set model that has learned the priority tendency of each doctor and each hospital, and the like.

Herein, the term “processor” used in the above explanation means, for example, circuitry such as a central processing unit (CPU), a graphics processing unit (GPU), an application-specific integrated circuit (ASIC), or a programmable logic device (e.g., a simple programmable logic device (SPLD), a complex programmable logic device (CPLD), or a field-programmable gate (FPGA). If the processor is a CPU, for example, the processor implements its functions by reading and executing the programs stored in the storage circuitry. On the other hand, if the processor is an ASIC, for example, its functions are directly incorporated into the circuitry of the processor as logic circuitry, instead of a program being stored in the storage circuitry. The embodiment described herein does not limit each processor to a single circuitry-type processor. Multiple independent circuits may be combined and integrated as one processor to realize the intended functions. Furthermore, the multiple components shown in the figures may be integrated into a single processor to implement its functions.

In addition, the functions described in the above embodiment may be implemented by installing a program for executing the processing in a computer, such as a workstation, and expanding the program in a memory. The programs that can cause the computer to execute the processing can be stored in a storage medium, such as a magnetic disk (a hard disk, etc.), an optical disk (CD-ROM, DVD, etc.), or a semiconductor memory, and be distributed through it.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical information processing apparatus comprising processing circuitry configured to:
acquire patient information relating to a target patient, environment information relating to an environment for execution, and target data relating to the target patient; and
determine, from a plurality of trained models differing from each other in processing speed and accuracy, a combination of trained models that satisfies a condition according to the patient information and the environment information.

2. The medical information processing apparatus according to claim 1, wherein
the plurality of trained models are prepared for each of one or more analytical processing steps, and
if a task that includes a plurality of analytical processing steps is to be performed, the processing circuitry selects one trained model from the prepared plurality of trained models for each analytical processing step to determine the combination.

3. The medical information processing apparatus according to claim 1, wherein the processing circuitry determines the combination based on whether to prioritize the processing speed or the accuracy.

4. The medical information processing apparatus according to claim 1, wherein
the patient information includes vital information of the target patient, and
the environment information includes at least one of a system environment for executing a trained model or a scale of a hospital.

5. The medical information processing apparatus according to claim 1, wherein the processing circuitry is further configured to display information relating to an analytical processing step in progress.

6. The medical information processing apparatus according to claim 5, wherein the processing circuitry is further configured to display information of a selected trained model for each analytical processing step.

7. The medical information processing apparatus according to claim 5, wherein the processing circuitry is further configured to calculate a processing time and estimation accuracy that are assumed for the trained model,
wherein the processing circuitry displays the trained model and at least one of the processing time or the estimation accuracy in such manner that the trained model is associated with the at least one of the processing time or the estimation accuracy.

8. The medical information processing apparatus according to claim 1, wherein the processing circuitry is further configured to:
train a network model using, as input data, information regarding the trained models included in the combination, the patient information, the environment information, and the target data, and using, as ground truth data, feedback information relating to feedback from a user on at least one of a state during execution of an analytical processing step using the combination, or an analysis result; and
generate a trained set model.

9. The medical information processing apparatus according to claim 8, wherein the processing circuitry is further configured to input the patient information, the environment information, and the target data, to the trained set model and thereby output the combination of trained models.

10. A medical information processing method, comprising:
acquiring patient information relating to a target patient, environment information relating to an environment for execution, and target data relating to the target patient; and
determining, from a plurality of trained models differing from each other in processing speed and accuracy, a combination of trained models that satisfies a condition according to the patient information and the environment information.

11. A non-transitory computer-readable medium having recorded thereon a plurality of computer-executable instructions that cause the computer to execute the steps of:
acquiring patient information relating to a target patient, environment information relating to an environment for execution, and target data relating to the target patient; and
determining, from a plurality of trained models differing from each other in processing speed and accuracy, a combination of trained models that satisfies a condition according to the patient information and the environment information.
